(19) United States
(12) Patent Application Publication
(10) Pub. No.: US 2013/0345557 A1
(43) Pub. Date: Dec. 26, 2013

(54) LIGHT SCANNING PROBE AND MEDICAL IMAGING APPARATUS EMPLOYING THE SAME

(71) Applicant: SAMSUNG ELECTRONICS CO., LTD., Suwon-si (KR)

(72) Inventors: Jong-seok KIM, Hwaseong-si (KR); Sung-chan KANG, Hwaseong-si (KR); Woon-bae KIM, Seoul (KR); Yong-seop YOON, Seoul (KR); Hyung CHOI, Seongnam-si (KR)

(21) Appl. No.: 13/927,655

(22) Filed: Jun. 26, 2013

(30) Foreign Application Priority Data
Jun. 26, 2012 (KR) 10-2012-0068705

(51) Int. Cl. A61B 5/00; G02B 26/12; G02B 26/10

(52) U.S. Cl. CPC A61B 5/0095; G02B 26/10; G02B 26/125
USPC 600/425; 359/205.1

(57) ABSTRACT

A light scanning probe includes: a probe main body; a light scanner that includes a scanner module that is driven to rotate and a beam reflector that includes a plurality of reflection surfaces which alter a path of light being scanned by the scanner module, wherein the light scanner is disposed within the probe main body; and an optical fiber that guides light which is received from a light input unit toward the scanner module. A medical imaging apparatus includes the light scanning probe that irradiates light toward an object; a receiver that receives a signal which is generated in the object; and a signal processor that processes the signal received by the receiver.

### CROSS-REFERENCE TO RELATED APPLICATIONS

This application claims priority from Korean Patent Application No. 10-2012-0068705, filed on Jun. 26, 2012, in the Korean Intellectual Property Office, the disclosure of which is incorporated herein by reference in its entirety.

### BACKGROUND

1. Field

Exemplary embodiments described herein relate to light scanning probes and medical imaging apparatuses employing the same.

A 2. Description of the Related Art

The demand for technologies for performing precise tomography on lower layers of human skin tissue and the demand for information which relates to human skin tissue in the field of medical imaging are increasing. In particular, because most cancers start in lower cells of the epithelium and are spread into cells of the hypodermis, in which blood vessels exist, if early-stage cancer can be detected, injury caused by cancer can be considerably reduced. In existing imaging technologies, such as magnetic resonance imaging (MRI), x-ray computed tomography (CT), ultrasonography, and the like, tomography may be performed on layers inside human skin tissue while penetrating human skin tissue. However, because resolutions of devices for performing such imaging technologies are relatively low, early-stage cancer, in which a tumor is small, may not be detected. Conversely, optical coherence tomography (OCT), optical coherence microscopy (OCM), and photoacoustic tomography (PAT) technologies that have been recently introduced use light. Thus, although penetration depths of light into skin that are in the range of 1 to 2 mm (OCT) and 30 to 50 mm (PAT), are lower than those of existing imaging methods, the resolutions of devices for performing these technologies are about 10 times higher than those of ultrasound devices. Thus, these techniques may be efficiently used for performing early-stage cancer diagnosis.

When such medical imaging techniques using light are applied to performing an internal diagnosis of the human body, for example, using any of an endoscope, a laparoscope, and robotic surgery, a light probe is used to receive light from a light source and send the light into the human body. Various scanning methods are used in conjunction with the light probe in order to obtain an image of a predetermined region of an object to be examined. For example, a method for using a bunch of optical fibers, a method for controlling a light path by directly modifying an optical fiber, and a method for splitting a light path by using a plurality of beam splitters are used.

### SUMMARY

Provided are light scanning probes that irradiate light while scanning a predetermined region of an object to be examined and medical imaging apparatuses employing the same.

Additional aspects will be set forth in part in the description which follows and, in part, will be apparent from the description, or may be learned by practice of the presented exemplary embodiments.

According to an aspect of one or more exemplary embodiments, a light scanning probe includes: a probe main body that has an inner space, wherein a first end of the probe main body functions as a light input unit and a second end of the probe main body functions as a light output unit; a light scanner that includes a scanner module that is driven to rotate and a beam reflector that includes a plurality of reflection surfaces which alter a path of light being scanned by the scanner module, wherein the light scanner is disposed within the probe main body in correspondence with the light output unit; and an optical fiber that guides light which is received by the light input unit toward the scanner module.

The plurality of reflection surfaces may be disposed to reflect lights incident in different directions as the scanner module rotates toward an outside of the light scanning probe.

The plurality of reflection surfaces may be respectively disposed at respective angles such that the lights incident in different directions as the scanner module rotates are reflected in a same direction.

The beam reflector may include a plurality of reflection members.

At least one of the plurality of reflection members may include one of a mirror and a prism.

The plurality of reflection surfaces may be integrally connected to each other.

The scanner module may include a micromirror.

The scanner module may include a rotating polygon mirror, and a lens may be disposed between the scanner module and the beam reflector.

The light scanning probe may further include a transparent beam guide that fixes the beam reflector in the probe main body.

The light scanning probe may further include a power sensor that is disposed in a path of light reflected by the scanner module and that measures a power of the light which is received by the light input unit.

According to another aspect of one or more exemplary embodiments, a medical imaging apparatus includes: a light source; the light scanning probe which irradiates light which is received from the light source toward an object to be examined while scanning the object; a receiver that receives a signal generated in the object; and a signal processor that processes the signal received by the receiver and generates an image signal.

The light source may include a pulsed laser that induces ultrasonic waves from the object.

The receiver may include a transducer that converts the ultrasonic waves generated in the object to an electrical signal.

The light scanner may further include a power sensor that is disposed in a path of light reflected by the scanner module and that measures a power of the light which is received from the light source, and the medical imaging apparatus may further include a controller that receives a feedback from the power sensor and controls the power of the light source.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 schematically shows a light scanning probe, according to an exemplary embodiment;

FIGS. 2A, 2B, 2C, and 2D are diagrams which illustrate a light scanning operation of the light scanning probe of FIG. 1;

FIG. 3 schematically shows a light scanning probe, according to another exemplary embodiment;

FIG. 4 schematically shows a light scanning probe, according to yet another exemplary embodiment;

FIG. 5 schematically shows a light scanning probe, according to still another exemplary embodiment;

FIG. 6 schematically shows a light scanning probe, according to yet another exemplary embodiment; and

FIG. 7 is a schematic block diagram of a medical imaging apparatus, according to an exemplary embodiment.

### DETAILED DESCRIPTION

Reference will now be made in detail to exemplary embodiments, examples of which are illustrated in the accompanying drawings, wherein like reference numerals refer to the like elements throughout. In this regard, the present exemplary embodiments may have different forms and should not be construed as being limited to the descriptions set forth herein. Accordingly, the exemplary embodiments are merely described below, by referring to the figures, in order to explain aspects of the present disclosure.

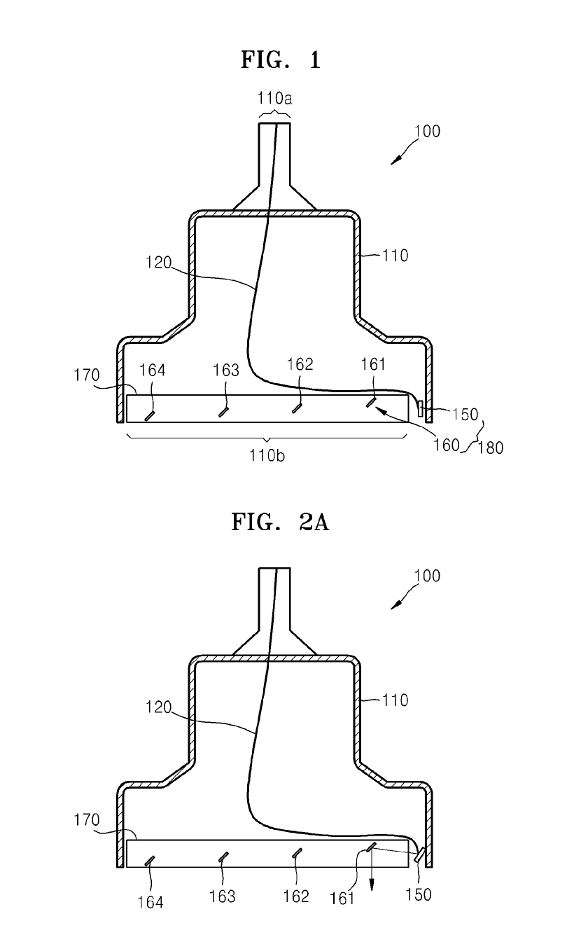

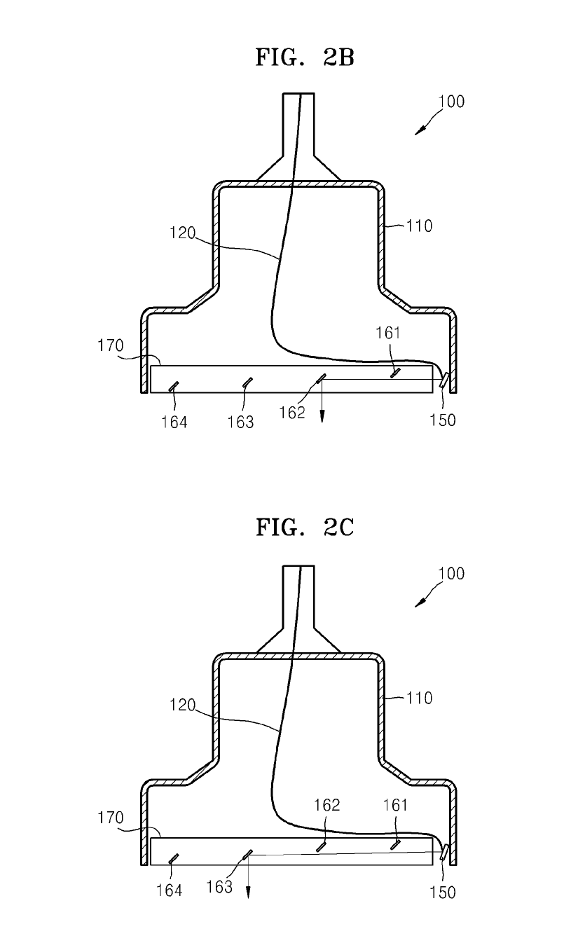

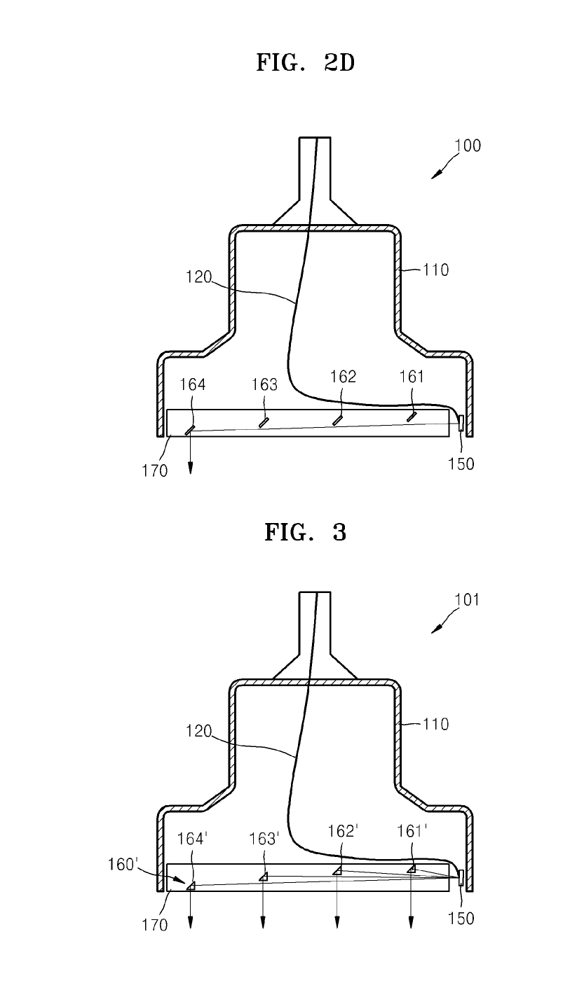

FIG. 1 schematically shows a light scanning probe 100, according to an exemplary embodiment. FIGS. 2A, 2B, 2C, and 2D are diagrams which illustrate a light scanning operation of the light scanning probe 100 of FIG. 1.

The light scanning probe 100 includes a probe main body 110, and a light scanner 180 and an optical fiber 120 which are disposed in the probe main body 110.

The probe main body 110 has an inner space and a shape in which one end of the probe main body 110 functions as a light input unit 110a and in which the other end of the probe main body functions as a light output unit 110b. In particular, the light input unit 110a may be connected to an external light source, and light that is received from the external light source propagates via the optical fiber 120 and the light scanner 180, and is emitted from the light output unit 110b.

The light scanning probe 100 irradiates light used in conjunction with practicing a medical imaging technique for performing a diagnosis toward a predetermined region in the human body at a uniform intensity.

For this purpose, the light scanner 180 is disposed within the probe main body 110 in correspondence with the light output unit 110b, and includes a scanner module 150 that is driven to rotate and a beam reflector 160 that includes a plurality of reflection surfaces that alter a path of light which is scanned by the scanner module 150.

The optical fiber 120 guides light which is received by the light input unit 110a toward the scanner module 150.

The scanner module 150 may be a movable micromirror as shown herein, and scans light transmitted via the optical fiber 120 in another direction as the scanner module 150 is rotated by a driving source (not shown). Various driving sources such as a galvano motor and piezo actuator may be used to drive the micromirror.

The beam reflector 160 may include a plurality of reflection members, for example, a first reflection member 161, a second reflection member 162, a third reflection member 163, and a fourth reflection member 164, as shown herein. The first to fourth reflection members 161, 162, 163, and 164 may be fixed in a beam guide 170 which is formed of a transparent material, such as, for example, glass or transparent plastic.

The first to fourth reflection members 161, 162, 163, and 164 may include mirrors, each of which includes a reflection surface that reflects incident light. The reflection surface is disposed to reflect lights that are incident in different directions as the scanner module 150 rotates and to emit the lights to the outside of the light scanning probe 100. In addition, for example, each of the reflection surfaces may be respectively aligned at a respective angle so as to reflect lights incident in different directions as the scanner module 150 rotates and to emit the reflected lights in the same direction.

Referring to FIG. 2A, the scanner module 150 is disposed such that light incident from the optical fiber 120 is reflected toward the first reflection member 161. The first reflection member 161 alters the path of the light, thereby causing the reflected light to be emitted toward the outside of the light scanning probe 100.

Referring to FIG. 2B, the scanner module 150 is disposed such that light incident from the optical fiber 120 is reflected toward the second reflection member 162, and the second reflection member 162 alters the path of the light, thereby causing the reflected light to be emitted toward the outside of the light scanning probe 100.

Referring to FIGS. 2C and 2D, the scanner modules 150 are respectively disposed to irradiate lights toward the third reflection member 163 and the fourth reflection member 164, respectively, and the third reflection member 163 and the fourth reflection member 164 alter the paths of the incident lights, thereby causing the reflected lights to be emitted the outside of the light scanning probe 100.

Although the beam reflector 160 includes four reflection members in the foregoing example, the number of reflection members may be modified in various ways. In addition, although the scanner module 150 rotates in one direction in the foregoing example, the scanner module 150 may be driven in two axes to have various scanning directions.

FIG. 3 schematically shows a light scanning probe 101, according to another exemplary embodiment.

The current exemplary embodiment is different from the previous exemplary embodiment in that a beam reflector 160′ of the scanning probe 101 includes prism-shaped first to fourth reflection members 161′, 162′, 163′, and 164′. In particular, the first to fourth reflection members 161′, 162′, 163′, and 164′ alter paths of incident lights by total reflection due to difference in refractive index.

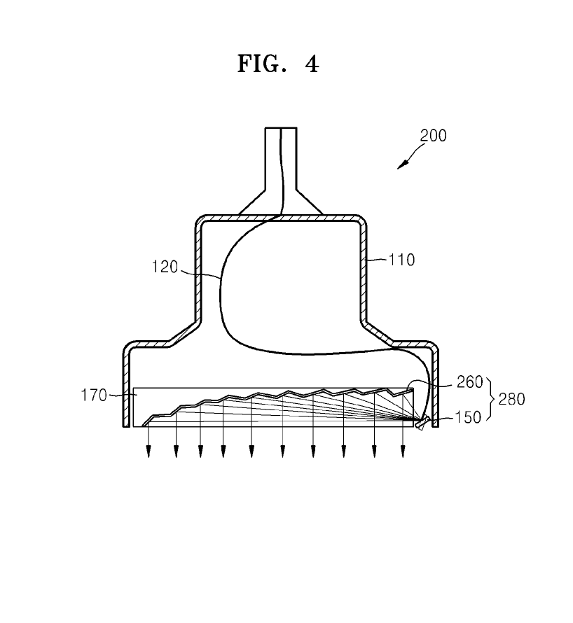

FIG. 4 schematically shows a light scanning probe 200, according to yet another exemplary embodiment.

The current exemplary embodiment is different from the previous exemplary embodiments in that a beam reflector 260 of a light scanner 280 includes a plurality of reflection surfaces which are integrally connected to each other. In this regard, for example, when a pulsed light is incident onto the scanner module 150 from the optical fiber 120, there is a margin in controlling spotting time. The reflection surfaces of the beam reflector 260 may be mirror surfaces, or may use total reflection by difference in refractive index. The number of reflection surfaces of the beam reflector 260 and the inclination of the reflection surfaces may be modified in various ways. The location of the scanner module 150 may be controlled in consideration of the relation with the beam reflector 260.

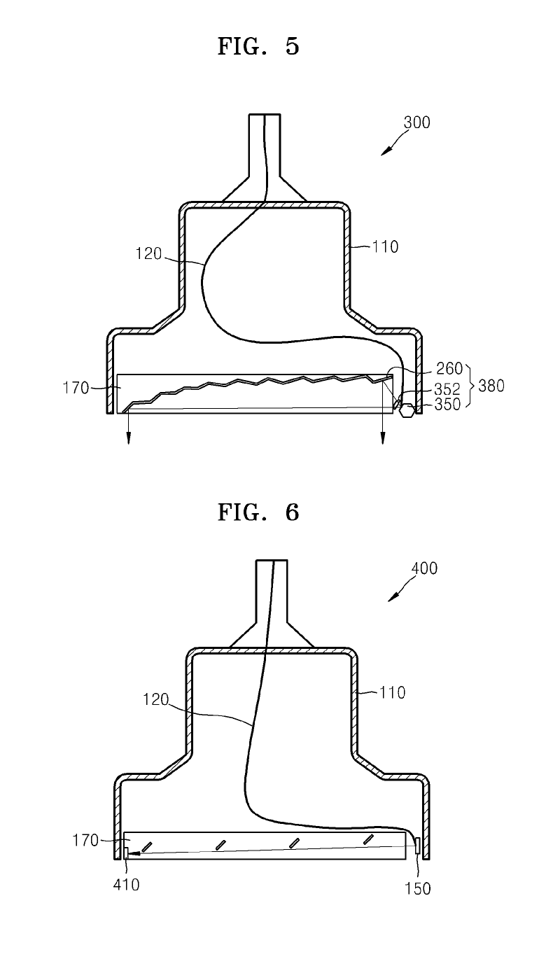

FIG. 5 schematically shows a light scanning probe 300, according to still another exemplary embodiment.

The current exemplary embodiment is different from the previous exemplary embodiments in that a light scanner 380 includes a scanner module 350 having a rotating polygon mirror shape that includes a plurality of reflection surfaces. A lens 352 may further be disposed in a light path between the scanner module 350 and the beam reflector 260.

FIG. 6 schematically shows a light scanning probe 400, according to yet another exemplary embodiment.

The current exemplary embodiment is different from the previous exemplary embodiments in that the light scanning probe 400 further includes a power sensor 410 that is disposed in the path of light reflected by the scanner module 150 and that measures a power of incident light. The information measured by the power sensor 410 may be sent to a light source that provides light to be input to the optical fiber 120 as a feedback. FIG. 6 shows the light scanning probe 100 of FIG. 1 as further including the power sensor 410. However, the power sensor 410 may also be applied to the light scanning probes 101, 200, and 300 of FIGS. 3, 4, and 5. In addition, the location of the power sensor 410 is not limited, and the power sensor 410 may be disposed at any position in the path of the light reflected by the scanner module 150.

The light scanning probe described above, which has a structure which includes a driving scanner and a beam reflector, is simpler and more compact than a structure which relies upon a bunch of optical fibers or a structure which relies upon a plurality of beam splitters, and performs a scanning by efficiently distributing externally input light in a predetermined region of an object to be examined.

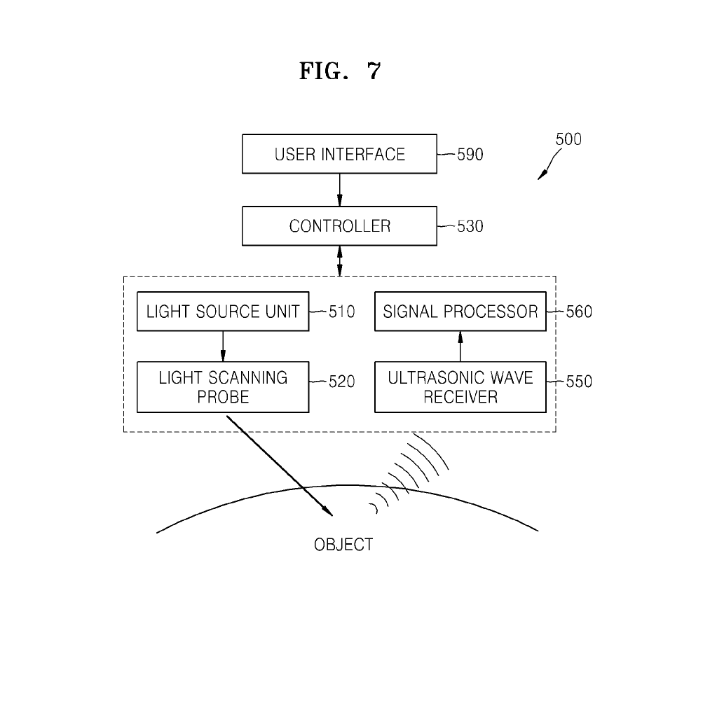

FIG. 7 is a schematic block diagram of a medical imaging apparatus 500, according to an exemplary embodiment.

The medical imaging apparatus 500 includes a light source 510, a light scanning probe 520 that irradiates light received from the light source 510 in order to scan an object to be examined, a receiver 550 that receives a signal generated from the object, and a signal processor 560 that processes the signal received by the receiver 550.

The medical imaging apparatus 500 uses a photoacoustic tomography (PAT), which is a technique for performing imaging by sensing pressure waves generated by laser pulses in cell tissues of an object to be examined. When laser beams are irradiated toward a liquid or solid material, the material absorbs optical energy in order to immediately generate thermal energy that generates acoustic waves by a thermoelastic phenomenon. Because an absorption rate which relates to the wavelength of light and a thermoelastic coefficient may vary based on the material constituting the object, lights having the same energy may generate ultrasonic waves of different amplitudes. By detecting the ultrasonic waves, images of the distribution of blood vessels and minute changes of tissue in the human body may be obtained by non-invasive methods.

The light source 510 may irradiate a pulsed laser that induces ultrasonic waves from the object, and a pulse width may be in a range of between approximately several pico-seconds and approximately several nano-seconds.

The light scanning probe 520 irradiates light while scanning a predetermined region of an object to be examined, and may include, for example, at least one of the light scanning probes 100, 101, 200, 300, and 400, or any combination thereof.

When light is irradiated by the light scanning probe 520 toward the object, ultrasonic waves are generated in the object. The ultrasonic waves may have different frequency bands or different amplitudes, based on one or more of a pulse width of a laser beam, a pulse fluence of a laser beam, and a laser absorption coefficient, a laser reflection coefficient, specific heat, and a thermal expansion coefficient of the object. In particular, when a laser beam is irradiated toward the object, different ultrasonic waves are generated, based on the type of the object. By detecting the ultrasonic waves, images which may be used to determine the type of the object may be obtained.

The receiver 550 may be an ultrasonic wave receiver, and may include, for example, a transducer that converts the ultrasonic wave generated in the object into an electrical signal. For example, the transducer may include a piezoelectric micromachined ultrasonic transducer (pMUT) that converts vibration caused by ultrasonic waves into an electrical signal. The pMUT may include at least one of a piezoelectric ceramic having a piezoelectric property, a single crystalline, and a composite piezoelectric material including at least one of the piezoelectric ceramic and the single crystalline, and a polymer. The transducer may include at least one of a capacitive micromachined ultrasonic transducer (cMUT), a magnetic micromachined ultrasonic transducer (mMUT), and an optical ultrasonic detector.

The signal processor 560 may process the signal received from the receiver 550 in order to generate an image signal.

In addition, the medical imaging apparatus 500 may further include a user interface 590 and a controller 530. The user interface 590 may include an input unit and a display unit, and may provide a required input to the controller 530 by using either or both of these units.

The controller 530 controls each element of the medical imaging apparatus 500 based on a command which is received from the user interface 590. For example, the controller 530 may control the operation of the scanner module of the light scanning probe 520. In addition, the controller 530 may control the intensity of light of the light source 510 such that light with uniform intensity may be irradiated toward the object based on the feedback signal measured by the power sensor of the light scanning probe 520. The controller 530 may include a microprocessor.

Although the medical imaging apparatus which uses PAT is described herein, the light scanning probe 520 may also be applied to a medical imaging apparatus which uses OCT or OCM. In this regard, the detection sensor which is included in the receiver may vary based on the type of the signal generated in the object, and the receiver may be disposed within the light scanning probe 520.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

What is claimed is:

1. A light scanning probe, comprising:
a probe main body that has an inner space, wherein a first end of the probe main body functions as a light input unit and a second end of the probe main body functions as a light output unit;
a light scanner that comprises a scanner module that is driven to rotate and a beam reflector that comprises a plurality of reflection surfaces which alter a path of light being scanned by the scanner module, wherein the light scanner is disposed within the probe main body in correspondence with the light output unit; and
an optical fiber that guides light which is received by the light input unit toward the scanner module.

2. The light scanning probe of claim 1, wherein the plurality of reflection surfaces are disposed to reflect lights incident in different directions as the scanner module rotates toward an outside of the light scanning probe.

3. The light scanning probe of claim 2, wherein the plurality of reflection surfaces are respectively disposed at respective angles such that the lights incident in different directions as the scanner module rotates are reflected in a same direction.

4. The light scanning probe of claim 2, wherein the beam reflector comprises a plurality of reflection members.

5. The light scanning probe of claim 4, wherein at least one of the plurality of reflection members comprises one of a mirror and a prism.

6. The light scanning probe of claim 1, wherein the scanner module comprises a micromirror.

7. The light scanning probe of claim 2, wherein the plurality of reflection surfaces are integrally connected to each other.

8. The light scanning probe of claim 7, wherein the scanner module comprises a rotating polygon mirror.

9. The light scanning probe of claim 8, wherein a lens is disposed between the scanner module and the beam reflector.

10. The light scanning probe of claim 1, further comprising a transparent beam guide that fixes the beam reflector in the probe main body.

11. The light scanning probe of claim 1, further comprising a power sensor that is disposed in a path of light reflected by the scanner module and that measures a power of the light which is received by the light input unit.

12. A medical imaging apparatus comprising:
a light source;
the light scanning probe of claim 1 which irradiates light which is received from the light source toward an object to be examined while scanning the object;
a receiver that receives a signal generated in the object; and
a signal processor that processes the signal received by the receiver and generates an image signal.

13. The medical imaging apparatus of claim 12, wherein the light source comprises a pulsed laser that induces ultrasonic waves from the object.

14. The medical imaging apparatus of claim 13, wherein the receiver comprises a transducer that converts the ultrasonic waves generated in the object to an electrical signal.

15. The medical imaging apparatus of claim 13, wherein the light scanner further comprises a power sensor that is disposed in a path of light reflected by the scanner module and that measures a power of the light which is received from the light source.

16. The medical imaging apparatus of claim 15, further comprising a controller that receives a feedback from the power sensor and controls the power of the light source.
